(19) United States
(12) Patent Application Publication
(10) Pub. No.: US 2023/0115223 A1
(43) Pub. Date: Apr. 13, 2023

(54) EMERGENCY USE VENTILATOR

(71) Applicants: CHAN ZUCKERBERG BIOHUB, INC., San Francisco, CA (US); THE BOARD OF TRUSTEES OF THE LELAND STANFORD JUNIOR UNIVERSITY, Stanford, CA (US)

(72) Inventors: David B. Camarillo, Stanford, CA (US); Ryan Van Wert, San Francisco, CA (US); Samuel J. Raymond, Stanford, CA (US); Trevor Wesolowski, Oakland, CA (US); Larry Miller, Scotts Valley, CA (US)

(21) Appl. No.: 17/913,677

(22) PCT Filed: Mar. 26, 2021
(86) PCT No.: PCT/US21/24298

§ 371 Date: Sep. 22, 2022

Related U.S. Application Data
(60) Provisional application No. 63/081,144, filed on Sep. 21, 2020.
(60) Provisional application No. 63/010,517, filed on Apr. 15, 2020.
(60) Provisional application No. 62/994,856, filed on Mar. 26, 2020.

(51) Int. Cl. A61M 16/20; A61M 16/00; A61M 16/12

(52) U.S. Cl. CPC A61M 16/204; A61M 16/0069; A61M 16/125; A61M 16/205; A61M 2016/0042

(57) ABSTRACT

A ventilator system configured to switch between one or more invasive ventilation modes and one or more non-invasive ventilation modes is provided, the ventilator system comprising: an externally pressurized source of pre-mixed gas comprising air and oxygen; one or more inspiratory valves configured to deliver incoming pre-mixed gas to a patients breathing circuit; and one or more expiratory valves configured to remove outgoing gas from the patients breathing circuit; wherein in the one or more invasive ventilation modes, the inspiratory valves and the expiratory valves are configured to open and close to allow or prevent the passage of gas as needed in order to enforce a respiration cycle within the patient; and wherein in the one or more non-invasive ventilation modes, the inspiratory valves and the expiratory valves are kept open in order to allow the gas to pass freely through the system.

### CROSS-REFERENCE TO RELATED APPLICATIONS

The present application claims priority to U.S. Provisional Patent Application No. 62/994,856, filed Mar. 26, 2020, and entitled “Low Cost Mechanical Ventilator;” U.S. Provisional Patent Application No. 63/010,517, filed Apr. 15, 2020, and entitled “Ventilator Flow Rate Table;” and U.S. Provisional Patent Application No. 63/081,144, filed Sep. 21, 2020, and entitled “Emergency Use Ventilator,” the entirety of each of which are incorporated by reference herein.

### FIELD OF THE DISCLOSURE

The present disclosure generally relates to an emergency use ventilator.

### BACKGROUND

COVID-19 is highly contagious and can lead to respiratory distress, severe hypoxemia, and respiratory failure. The World Health Organization estimates that 1 in 5 adults who contract the disease will require hospitalization for breathing difficulties, and 1 in 20 will receive care in the Intensive Care Unit (ICU) for respiratory failure and mechanical ventilation.

In the presence of a highly contagious and virulent virus, as is the case in the present pandemic, the need for hospital care, specialized equipment and skilled care providers, even in resource rich countries, can far outpace capacity. With limited equipment and skilled personnel, care is compromised and both morbidity and mortality increase substantially. During the covid-19 pandemic of the past 14 months, communities across the globe have confronted profound resource shortages and loss of life. Medical personnel were forced to make decisions about which lives most merited ongoing life support. In northern Italy, New York City, and areas of South America owing to shortages of equipment, prompted care givers to withhold care for patients with a low statistical probability of survival, with care withheld from many patients over 60 years of age.

Mechanical ventilators, devices that provide facilitate both oxygenation and ventilation, first became widely available in the 1950s. Initially, mechanical ventilators used time-cycled negative pressure to facilitate gas exchange, technology that addressed the respiratory insufficiency caused the polio virus. Subsequently, with more respiratory failure resulting from lung injury, positive pressure ventilation became more standard. As more insight into the pathophysiology of lung injury has accrued, superimposed on rapid advances in engineering, mechanical ventilators have become ever more sophisticated, expensive and maintenance intensive.

### SUMMARY

Respiratory failure complicates most critically ill patients with COVID-19 and is characterized by heterogeneous pulmonary parenchymal involvement, profound hypoxemia and pulmonary vascular injury. The high incidence of COVID-19 related respiratory failure has exposed critical shortages in the supply of mechanical ventilators, and those with the necessary skills to treat. Traditional mass-produced ventilators rely on an internal compressor and mixer to moderate and control the gas mixture delivered to a patient. However, the current emergency has energized the pursuit of alternative designs, enabling greater flexibility in supply chain, manufacturing, storage and maintenance considerations.

A low-cost ventilator designed and built in accordance with the Emergency Use guidance from the US Food and Drug Administration (FDA) is provided herein, wherein pressurized medical grade gases enter the ventilator and time limited flow interruption determines the ventilator rate and tidal volume. This strategy obviates the need for many components needed in traditional ventilators, thereby dramatically shortening the time from storage to clinical deployment, increasing reliability, while still providing physiologic ventilatory support.

The overall design philosophy and its applicability in this new crisis is first described, followed by both bench top and animal testing results used to confirm the precision, safety and reliability of this low cost and novel approach to mechanical ventilation. The ventilator meets and exceeds the critical requirements included in the FDA emergency use guidelines. The ventilator has received emergency use authorization from the FDA.

In an aspect, a ventilator system configured to switch between one or more invasive ventilation modes and one or more non-invasive ventilation modes is provided, the ventilator system comprising: an externally pressurized source of pre-mixed gas including air and oxygen; one or more inspiratory valves configured to deliver incoming pre-mixed gas to a patient's breathing circuit; and one or more expiratory valves configured to remove outgoing gas from the patient's breathing circuit; wherein in the one or more invasive ventilation modes, the inspiratory valves and the expiratory valves are configured to open and close to allow or prevent the passage of gas as needed in order to enforce a respiration cycle within the patient; and wherein in the one or more non-invasive ventilation modes, the inspiratory valves and the expiratory valves are kept open in order to allow the gas to pass freely through the system.

In another aspect, a method is provided, comprising: delivering, by a ventilator, incoming pre-mixed gas including air and oxygen to a patient's breathing circuit via one or more inspiratory valves of the ventilator; removing, by the ventilator, outgoing gas from the patient's breathing circuit via one or more expiratory valves of the ventilator; and switching, by the ventilator, between one or more invasive ventilation modes and one or more non-invasive ventilation modes; in the one or more invasive ventilation modes, opening and closing, by the ventilator, the inspiratory valves and the expiratory valves to allow or prevent the passage of gas as needed in order to enforce a respiration cycle within the patient; and in the one or more non-invasive ventilation modes, opening, by the ventilator, the inspiratory valves and the expiratory valves in order to allow the gas to pass freely through the system.

### BRIEF DESCRIPTION OF THE DRAWINGS

FIG. 1 illustrates a prototype of the new O2U ventilator, designed and built in the early months of the COVID-19 pandemic with accessories (top-left) an internal construction render (top-right) and the prototype schematic (bottom) showing the internal components (within the shaded region) and the relevant accessories and components required (non-shaded).

FIG. 2 illustrates positive end-expiratory pressure (PEEP) as a function of time as measured on the bench-test setup for the O2U ventilator. (Left) PEEP at a level of 5 cmH2O and (right) PEEP at 15 cmH2O set via an external diaphragm value on the ventilator.

FIG. 3 illustrates three graphs showing 10 (top), 15 (middle), and 20 (bottom) breaths per minute as set by the UI of the O2U ventilator for a flowrate of 50 L/min. Breathing rates control how rapidly air is supplied to the patient. Control of the breathing rate is essential for patient case.

FIG. 4 illustrates tidal volume in a range is shown for 50 mL increments from 300 mL to 500 mL. Tidal volume, or the volume delivered to the patient is a critical variable in patient care. The O2U ventilator is capable of providing the FDA-required ranges to manage the majority of COVID-19 patients.

FIG. 5 illustrates the difference in 1.0 s and 1.6 s inspiration time on the dynamic effects on the flow and PEEP pressure signals. Inspiration time is the duration of time that the air is delivered to the patient. This can be important to vary to manage different lung care strategies.

FIG. 6 illustrates the O2U ventilator performing mandatory ventilation on a sedated and intubated porcine subject.

FIG. 7 illustrates that during the animal test, while the flowrate was kept constant at 33 L/min, due to the changes in other operating parameters, the flow signals were different for each phase of the test.

FIG. 8 illustrates that while the PEEP pressure was maintained at 5 cmH2O for the animal test, during each phase the pressure peaks and durations changed in accordance with the ventilator setting and the animal response.

FIG. 9 illustrates that Delivered/Tidal volumes were the key variable in the animal test, where each phase consisted of a different volume to induce the measured responses in the animal that are shown in Table 4 and 5.

FIG. 10 illustrates X-Ray images of the animal lungs after the ventilator experiment. (left) Left Lateral Thorax, (middle) VD thorax, (right) Right Lateral Thorax.

FIG. 11 illustrates images of the animal's lungs after the experiment. Scale bars are 2 cm.

FIG. 12 illustrates a schematic of an exemplary mechanical ventilation device.

FIG. 13 illustrates an exemplary user interface for an exemplary mechanical ventilation device.

FIG. 14 illustrates a system model of an exemplary mechanical ventilation device.

FIG. 15 illustrates an exemplary system response to an exemplary mechanical ventilation device.

FIGS. 16 and 17 depicts isometric views of an exemplary mechanical ventilation device.

FIG. 18 illustrates a front view and flow paths of an exemplary mechanical ventilation device.

Table 1 illustrates Emergency Use Ventilator minimum requirements for treating COVID-19 patients.

Table 2 illustrates ventilator parameters used for benchtop testing as required for FDA EUA ventilator requirements as well as the animal ventilation test showing the phases of simulated ventilation treatment.

Table 3 illustrates measurement of blood gasses of the animal subject during the phases of simulated ventilation treatment.

Table 4 illustrates possible states of a main control valve and a diaphragm actuation valve of an exemplary mechanical ventilation device during inhale and exhale.

Table 5 illustrates a ventilator flow rate table.

### DETAILED DESCRIPTION

Given the profound shortage of resources, especially mechanical ventilators, this issue may be addressed by creating a safe, low-cost, rapid to manufacture and deploy ventilator, with sufficient functionality to provide both non- and invasive mechanical ventilation. Generally speaking, the present disclosure provides a device that can be safely stored over the long-term with the capacity for a rapid quality control check and deployment for patient care. With rapid changes in patient condition superimposed on the potential for highly contagious pathogens, the ventilator provided herein was designed to be capable of continuously delivering oxygen-rich air at flow rates as high as 45 liters per minute as used in High-flow Nasal Cannula and CPAP devices and positive pressure ventilation in both assisted and intermittent mandatory modes. To enable rapid and high volume manufacturing, the design of the ventilator provided herein minimizes the number of parts and severely limits the use of custom parts.

To address the critical shortage of mechanical ventilators (MV), the FDA provided explicit details surrounding the process and requirements for devices eligible for Emergency Use Authorization (EUA), which limited requirements to those vital to health and safety requirements. The EUA pathway authorized use of the for the duration of the emergency, with further regulatory clearance and FDA approval required upon the conclusion of the emergency. The EUA process was applicable to many product types in the fight against COVID-19, not just for ventilators. Relative to mechanical ventilators, the FDA provided explicit guidance for an engineering design in the Emergency Use Ventilator document. Table 1 shows these pertinent features. The functional, rapidly deployable, cost effective mechanical ventilator provided herein was created in view of this guidance, and the awareness that the typical supply chains would be pushed beyond capacity for ventilator-specific parts. For instance, the ventilator provided herein is powered using gases available in hospitals, and uses time-cycled flow interruption to for the breath rate and tidal volume.

The FDA offered guidance on the creation of 3 distinct ventilator categories. Specifically, the EUA offered guidance on Emergency Resuscitators, devices that provide positive pressure via mask or nasal interface, and emergency ventilators. The ventilator provided herein falls under the emergency ventilator category which entailed the capacity to deliver time-cycled, positive pressure breaths, limited by either pressure or volume,

The ventilator provided herein is able to be rapidly manufactured, relatively inexpensively, and can withstand long-term storage and be rapidly deployed to the patient care arena, and still reliably deliver time-cycled, precise gas volumes to patients. Furthermore, the ventilator provided herein supports spontaneously breathing patients via mask, nasal prongs or an endotracheal tube, as well as intubated patients with profound respiratory failure incapable of breathing spontaneously. This strategy allows a single ventilator to move with a patient through all phases of hospitalization which is especially important in the case of a highly contagious, fastidious viral pathogen such as COVID-19. To accomplish these goals, the number of parts used was minimized, readily available parts were used, and costs were minimized. The ventilator provided herein allows for ease of use and sufficient ventilatory flexibility to treat patients with mild, moderate or severe respiratory disease. The user interface is designed such that a provider familiar with fundamental principles of respiratory physiology and mechanical ventilation would be readily able to manage the device to facilitate rapid deployment and ready adoption, even when respiratory therapy, nursing and medical personnel are relatively unfamiliar with the ventilator itself.

First, the design of this ventilator is described including the design philosophy used to overcome the unique constraints in an EUA/COVID-19 situation, advantages and disadvantages of this approach are discussed. Next the device is shown to operate over the conditions expected during a full hospitalization using bench-top equipment to measure the relevant parameters from the ventilator. Finally a live animal experiment is presented and the results analyzed to determine the potential dangers of ventilation using this device to detect any causes of ventilator-induced lung injury, using a porcine subject. Finally, a discussion of the use of this ventilator platform and any relevant conclusions are presented.

### Materials and Methods

With the circumstances surrounding the emergency/rapid-response ventilator innovation period that occurred in the spring of 2020, certain design conditions were put in place both by the FDA regulations and by the nature of the emergency itself. This is the first time that the FDA has implemented an Emergency Use Authorization for ventilators. As such there were some unique differences between ventilators designed in line with the traditional 510(k) route and emergency use ventilators. The FDA streamlined the traditional 510(k) pathway for ventilators as there are several tests that take a significant amount of time (and money), which would preclude a rapid supply of ventilators available for approval and use. These are predominantly biocompatibility tests, which take months. An example of these were:
The FDA allowed the use of known (approved) materials to be used without requiring biocompatibility testing.
The FDA also reduced the electrical safety requirements the EUA ventilators need to meet down to those which are most important to patient and user safety and functionality (saving time and money).
   E.g. the requirement for an internal battery to power the ventilator was mitigated by allowing the use of an uninterruptible power supply.
To still ensure safety and functionality for the not required tests, the FDA relied on risk analyses and materials analysis to fill the testing gap.

An important note is that EUA approved ventilators can only be used while the EUA is in effect. Once the EUA is lifted, none of these EUA approved ventilators can be used or sold, even those in place at hospitals/care centers. So to continue using EUA ventilators thereafter, the manufacturer will have to complete all the 510(k) required testing, submit for and receive 510(k) clearance from the FDA.

A complete but scalable design was required to reach as many people as possible. Many different designs have been authorized by the FDA for the EUA and many will likely not be utilized. The ventilator provided herein is intended to exist after the EUA phase has passed and as such this design required some extra thought to its usefulness in the long term.

### Ventilator Design Philosophy

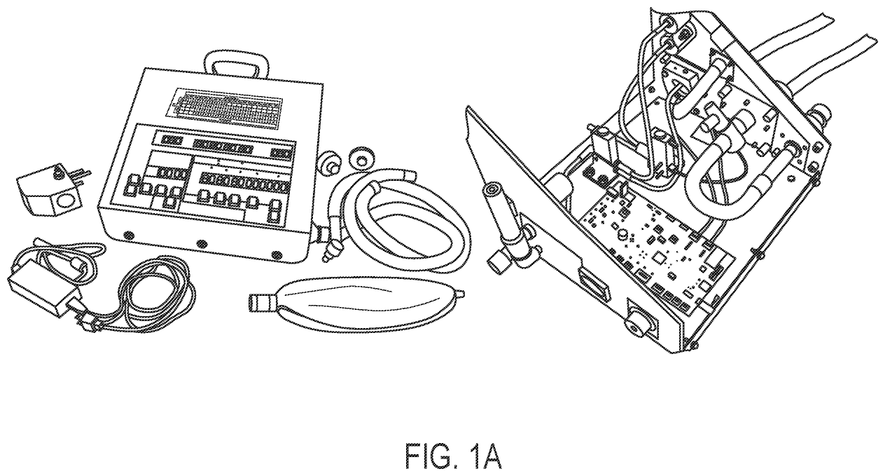

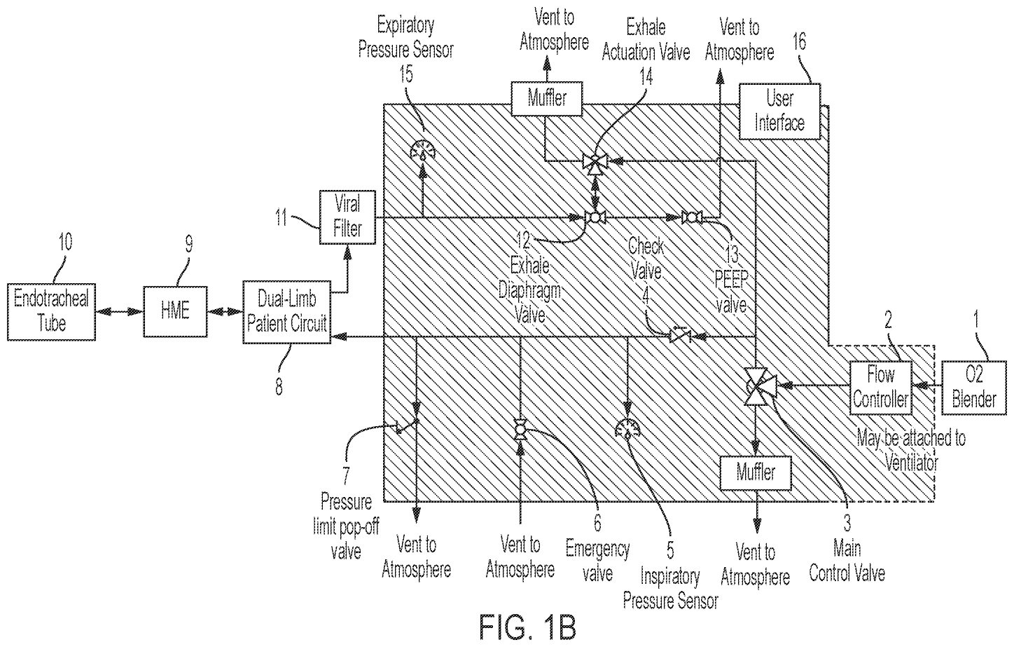

The ventilator provided herein is based on a continuous flow design, where the valves direct flow into or away from the patient's breathing circuit during invasive ventilation modes, and also allow for continuous flow during non-invasive mode operation. This is achieved by connecting the ventilator to pressurized sources of Air and Oxygen gas. These gasses are pre-mixed to the desired fraction of inspired Oxygen (FiO2) before entering the ventilator. During non-invasive operation modes this gas mixture is allowed to pass freely through the device, entering and exiting the patient breathing circuit, operating in the same manner as other CPAP or High Flow Nasal Cannula systems. Inspiratory and expiratory valves are kept open in this state and the system monitors the pressures in case of leaks or blockages or any other risks to patient and device safety. For invasive ventilation modes, either assisted or mandatory, the inspiratory and expiratory valves are used to allow or prevent the passage of incoming and outgoing gas, respectively, to enforce the respiration cycle within the patient. To overcome the lack of availability in flow-measuring sensors, this ventilator relies on a Pressure-Limited-Time-Cycled breathing loop where the flow rate is set on the ventilator using a manual control valve, also known as a Thorpe Tube, and the inspiratory time is set such that a known volume of gas is delivered during each inspiration phase. A specific tidal volume can then be delivered by knowing the flow rate and adjusting the inspiratory time such that the tidal volume (VT) can be calculated simply as VT=Flow rate x Inspiratory Time, where the operator can determine, for a user set flow rate and inspiratory time, the delivered flow volume. Volume calculations are still important for patient care, so in addition to this table for setting the desired delivered volume, a spirometer-based expiratory volume sensor is used on the expiration side of the circuit to measure exhaled volumes. The ventilator provided herein shown in FIG. 1.

### Advantages and Disadvantages

With any of the proposed ventilator designs that were published to meet this challenge, each of these approaches required compromising on some feature or price point in order to meet the best possible match of price to performance, however in addition to this, other constraints such as supply chain and clinical need meant that other choices were required that further added to the constraints and limitations for each design. The ventilator provided herein was designed using a number of unique choices that were done to maximize the utility of the ventilator to act as a one-device-one-visit platform. Below are two of the design choices that led to the most impactful differences between this design and the majority of other proposed ventilators.

### Reliance on Externally Pressurized and Mixed Gas

The ventilator provided herein relies on a pre-mixed and pressurized input of gas into the system. With the assumption being that most hospitals and care facilities will have access to both an Oxygen/Air blender and pressurized gasses, the benefit is that the ventilator provided herein can be made with far fewer parts and can utilize the pressure of incoming gas to pressurize the circuit. The input Thorpe tube regulates the pressure from wall pressures of typically 50 psi down to 1-3 psi, the regular range for patient breathing circuits.

### Pressure-Limited-Time-Cycled Ventilation

Most ventilators offer pressure-controlled or volume-controlled ventilation. This requires the use of a closed feedback look in the case of pressure-controlled case, or at least one flow sensor in the volume-controlled case. The ventilator provided herein operates in a modified manner compared to these two common cases and instead relies on a known flowrate entering the system and control of the valve timings. This control type: Pressure-Limited-Time-Cycled ensures that the pressures are always monitored to detect any leaks or obstructions that could risk patient or device safety, and that the desired delivered volume is instead controlled by the amount of time that the inspiration valve is open for. This assumption that the gas will be a constant input allows this ventilator to be used in non-invasive mode and invasive mode. The non-invasive case requires a mask be placed over the patient's mouth and the continuous flow is channeled through the mask and allowed to exit the expiratory valve, enabling the patient to take a breath of Oxygen-enriched/higher pressure gas that will lower the breathing effort. For the invasive ventilation where a patient is intubated, the timing of the valves directs the flow into or away from the patient. Tidal volume is set by allowing a known flowrate of gas to flow for a specified time.

A Single Ventilation Platform to Manage COVID-19 Patients

A typical patient who would suffer severe COVID-19 symptoms would follow a trajectory similar to that described as follows. A patient arrives at a hospital feeling weak and suffering from impaired breathing, but able to breathe on their own. They would normally present with low Oxygen saturation and be placed on a nasal cannula/CPAP or BiPAP machine delivering a higher concentration of Oxygen, potentially up to 100%. After some time the patient may continue to decline and become weaker and confused as the toll on their body leads to an increased work of breathing. The patient's lungs and airways have become compromised with mucus build up and general weakness, so the patient is placed under anesthesia and intubated to allow for a mechanical ventilator to be connected to assist in breathing, where a variety of assisted and mandatory modes may be used to properly manage the patient, at the discretion of the care provider. After such time as their body has been able to fight off the disease and the ventilator has protected their lungs as best as possible, weaning off the ventilator will occur and the patient will begin to take more spontaneous breaths. Eventually, before the patient is able to be extubated completely, they may be placed on a tracheal collar for passive Oxygen (still invasive). After additional time to build strength such that they could safely undergo extubation, they would likely require more non-invasive assistance for breathing, such as the High Flow Nasal Cannula or CPAP/BiPAP devices used at the beginning of their hospitalization. Finally, the patient would recover sufficiently to breathe completely unassisted, removed from all equipment, and subsequently be discharged. While this is not the case for every severity level of COVID-19 patients, this represents one such path that highlights the number of different breathing devices that may be required when being treated for this disease.

The ventilator provided herein simplifies this process and enables a single device to follow along with the patient as they progress with their treatment. This minimizes the number of different connections and devices that a medical professional needs to work with and maximizes the familiarity that the operator can have with the device which is important for those with less experience treating critically ill patients with ventilators. To show the abilities of this ventilator to manage these tasks a number of bench-top tests and animal testing were conducted to observe the flexibility of this ventilator in the cases where it was designed to be used. The animal test in particular was used to highlight the performance of the ventilator and any potential risk of damaging normal lungs.

Another key feature of the O2U ventilator is the rapid deployment afforded by its design. The process to take the ventilator from storage to usage requires the following steps:
1. Ensure the uninterruptible power supply (UPS) is fully charged (it should be stored charged and kept plugged in).
2. Unpack the ventilator—the gas pathway ports are capped off, so the gas pathway is clean and ready to use.
3. Visually inspect the chassis, flow meter, and labeling.
4. Remove the gas pathway port caps and connect a breathing circuit and the pressure monitoring line.
5. Place the test balloon on the breathing circuit.
6. Plug the UPS into the wall outlet and plug the ventilator into the UPS.
7. Turn the UPS on, which automatically initiates and runs the power on self test.
8. Run the alarms test.
9. Check for gas leakage in the breathing circuit
10. Put the ventilator in standby mode.
11. Set desired operating parameters.
12. Remove the test balloon and you are ready to connect to the patient ET tube or mask.

These steps can be performed quickly and with minimal training, important during high-stress situations when highly trained personnel are in short supply.

### Bench Top Testing—Protocol

To ensure that the ventilator provided herein met the EUA requirements needed to treat COVID-19 patients (any other aspects of FDA/regulatory compliance will not be discussed here) a number of tests were performed on the ventilator and the data recorded to ensure that these requirements could be met. Specifically, the following variables relevant to COVID-19 treatment were tested:
Positive Expiratory End Pressure (PEEP): to ensure that the lung always has some positive pressure inside so that the lung, which can become filled with fluid, does not collapse on itself during exhalation, a small amount of PEEP is often used to protect the lungs.
Breath Rate (breaths/min delivered): depending on the vitals of the patient, their lung characteristics, and the current state of the ventilator settings, different breath rates (how often the ventilator pushes air and Oxygen into the lung) are required.
Tidal Volume: Lung volumes typically relate to overall body size and hence the tidal volume (the total volume that the ventilator sends during inspiration) needs to be variable to treat patients of different ages, genders, and sizes.
Inspiratory Time (similar to I:E ratio): based on the breathing rate, a fixed amount of time is placed between breaths, this can be split into inspiration and exhalation phases at a ratio, normally more time is given for exhalation as the lungs contract slowly, resulting in a low expiratory flow rate compared to the ventilator driven inspiratory flow rate.
Tests were conducted using a Michigan Instruments test lung and two TSI flow measuring devices. The protocols for these parameter sweeps are outlined in Table 2. Results for the performance of the ventilator are shown in the next section.

### Live Animal Testing—Protocol

During ventilation, two types of injuries may be induced by mechanical ventilation: (a) high inspiration lung volume or pressure leading to alveolar overdistension, in which the lung tissue is damaged; (b) cyclic change in the non-aerated lung, for which the underlying cellular mechanism is still unclear. To prevent injury (a), tidal volume should be smaller than a critical value to avoid large stresses in the lung tissue. To prevent injury (b), PEEP should be set to avoid low end-expiratory lung volume (EELV), which will lead to high strains in the lung. The upper limit of the tidal volume will also help to avoid high strains, therefore it also helps to prevent the injury (b). To test this ventilator design and to add additional information beyond the minimum required by the EUA documents, a porcine animal study was conducted where a pig was ventilated under a number of conditions by the O2U ventilator in order to judge the potential for lung damage in the ventilator's operation. Pigs have been proven to be an effective large animal model for ventilation in previous work. Furthermore, humans and pigs have similar respiratory rates, which is an important parameter for replicating similar air circulation during breathing. The protocol for the animal test is shown in Table 2. This protocol was designed to both test the usability of this ventilator in a real-world scenario with a live subject, and to take the animal through various phases, representative of the cases that can occur in typical respiratory care where larger and smaller tidal volumes are prescribed during treatment. The animal used was a 65 Kg female pig with normal lungs. Arterial and venous blood lines were drawn from the animal periodically to monitor the effect of the ventilation during each phase of the experiment. A baseline of 10 mL/Kg of tidal volume was used as a baseline for this test with the hyper- and hypoventilation deviations based on 30% increase and reductions from this amount, respectively. Additionally, for this test, the animal's lungs were extracted after the experiment to observe any gross signs of injury. Results of this animal test are shown in the next section.

### Results

### EUA-Guided Bench Top Testing for Ventilator Specifications

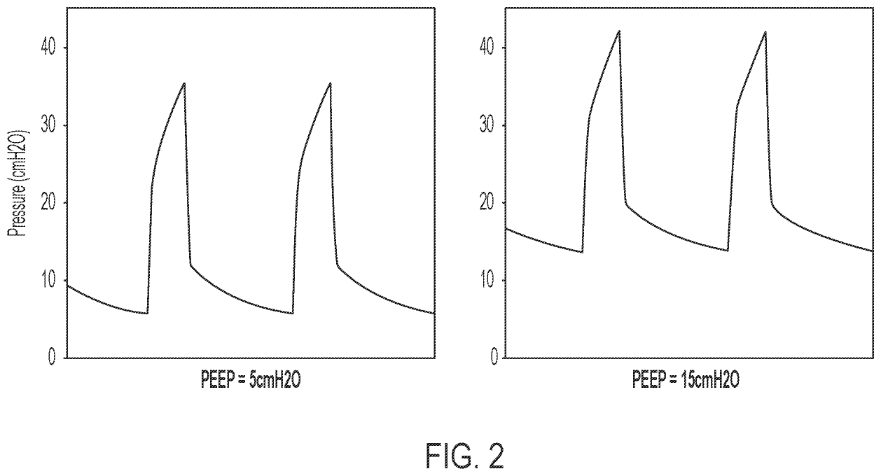

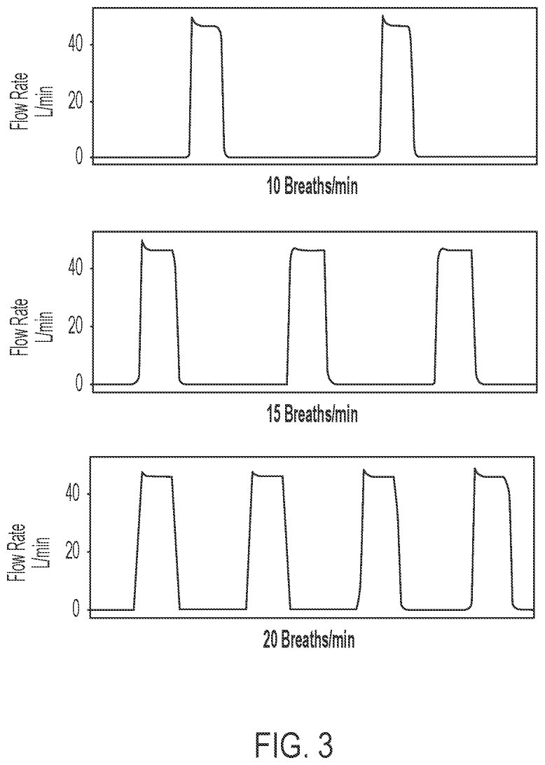

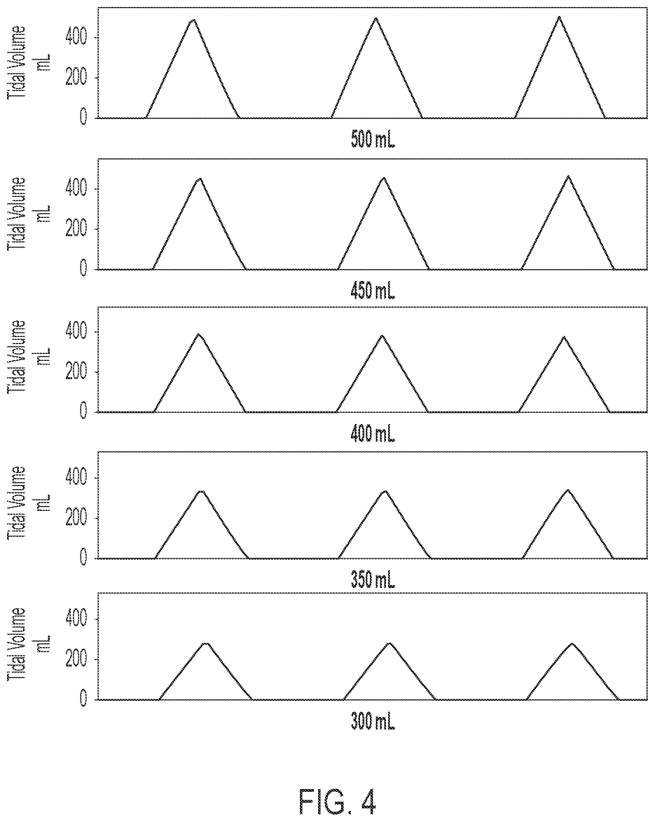

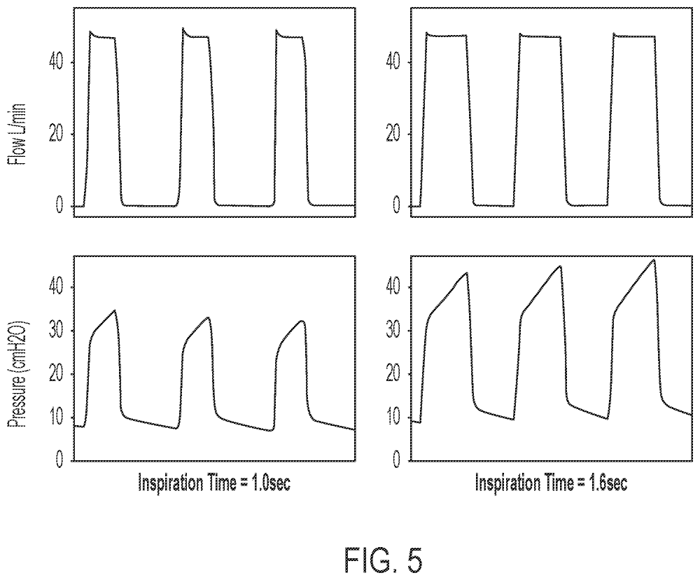

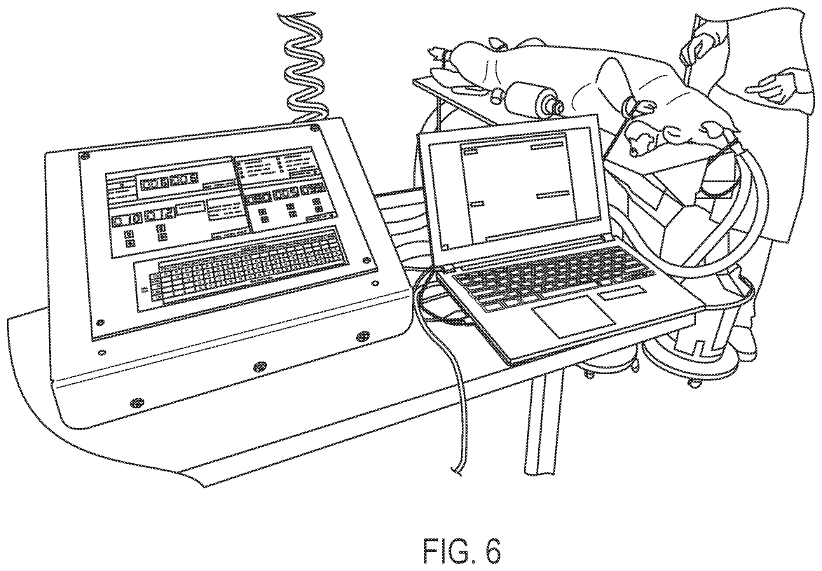

Data was recorded on the TSI flow meters and processed as comma separated value text files. These files were then post-processed using custom Python scripts to analyze the flow data. Flow of gasses were recorded, along with the pressure. These pressures were measured as absolute values and atmospheric pressure was measured during testing to calculate the gauge pressure, measured in cmH2O. All of the tests are listed in Table 2 and the analyzed data is shown in FIGS. 2-5 for PEEP, breathing rate, tidal volume, and inspiratory time, respectively. Data was recorded over several breaths for each test, representative plots are shown here for clarity.

### Porcine Ventilation Experiment for Ventilator Treatment Testing

The subject was sedated and intubated and immediately placed on the ventilator at the baseline rate of 10 breaths/min and 650 mL of tidal volume. Blood gasses were taken every 15 minutes, with the arterial gas and venous gas data shown in Table 3.

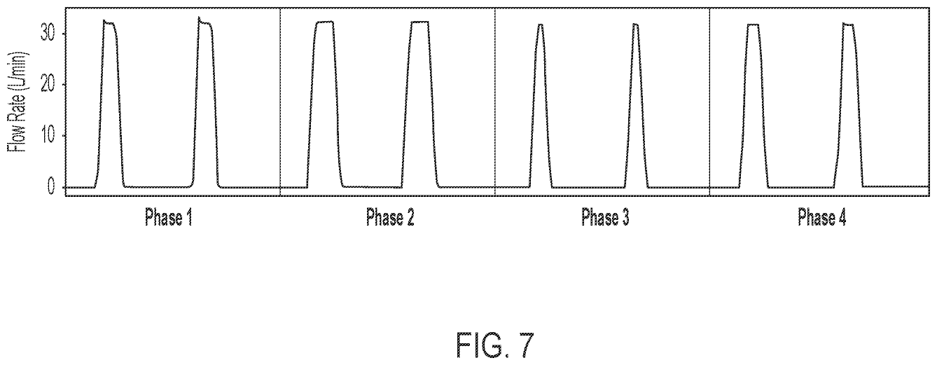

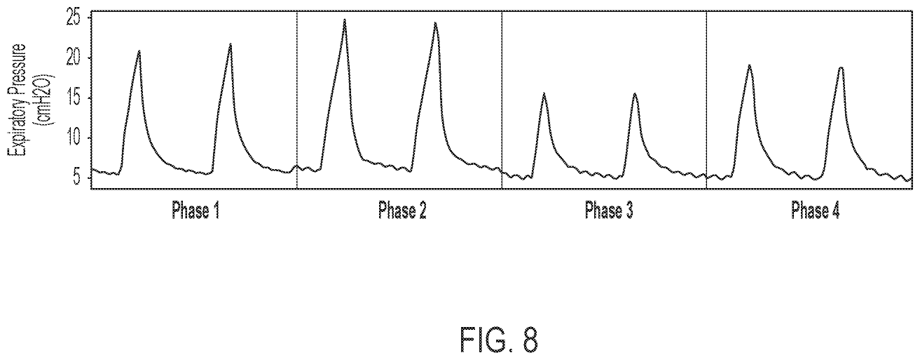

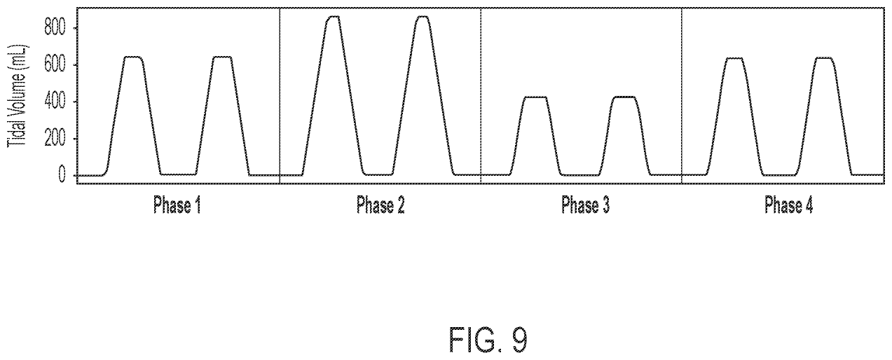

Data on the flow and pressure on the inspiratory side was measured using the same TSI flow meter used for the bench-top testing. Flow, pressure, and delivered volume for each phase listed in Table 2 are shown in FIGS. 7-9, respectively.

### Porcine Lung Histology

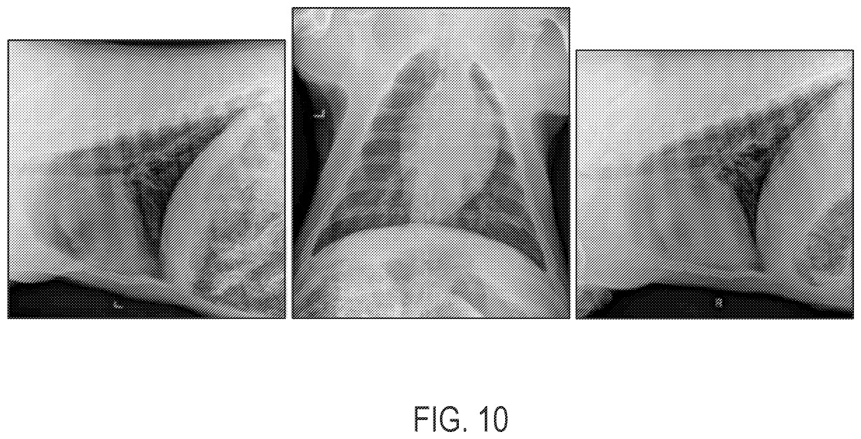

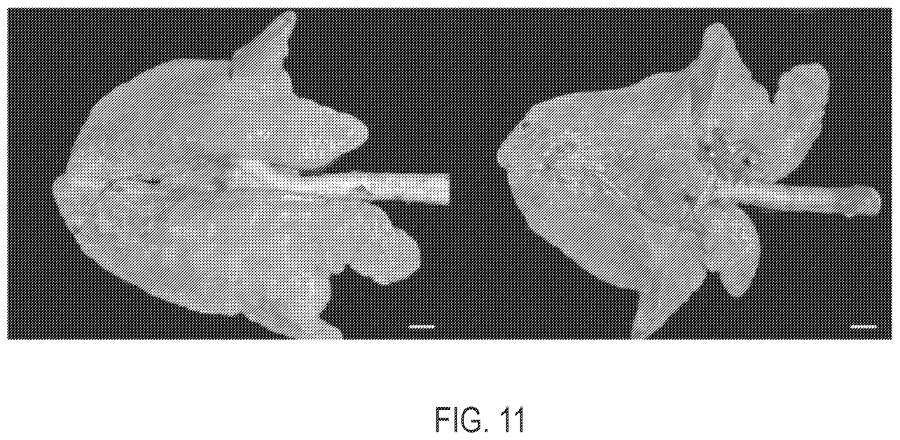

In addition to the blood gasses measured during the experiment, after the ventilation, the animal was euthanized and X-Rays and gross histology of the lungs were performed to ascertain the extent of potential damage caused by the ventilator. The X-Rays, shown in FIG. 10 and the histology report of the lungs indicate that there was some reddening/congestion in the caudal dorsal lung fields (see FIG. 11), which may have been positional as the pig was in dorsal recumbency during the procedure. There was no evidence of hyperinflation or atelectasis. These results indicate that the lungs were well preserved by the ventilator during the experiment.

### Discussion

The present disclosure illustrates the efficacy of a rapid-design, low-cost, versatile ventilator for use in crises such as the current COVID-19 pandemic. This design of the ventilator provided herein was based on a minimal component philosophy, utilizing the pressurized, medical grade, gasses to power the pneumatic breathing cycle. Timing of the gas delivery was controlled with valves and the monitoring of tidal volume ensured desired delivery. As this design resulted in an order of magnitude fewer components than conventional designs, safety and efficacy were crucial. Overall design efficacy and necessary features were governed by the adherence to the FDA's EUA requirements (see Table 1 for the recommendations and FIGS. 2-5 for the measured responses), allowing the device to be FDA approved. Additionally, a large animal experiment was also conducted to further understand and test the efficacy of the device. Both the fidelity in the desired and observed input parameters and the ease of use are critical factors in any emergency use device. A porcine subject was used to test the operation of the ventilator in achieving ventilation strategies that included under-ventilation, leading to observed hypoxic blood gasses, as well as over-ventilation, leading to hyperoxic reactions in the blood gasses. At a baseline of 650 mL tidal volume the animal was subjected to 30% increases and decreases to the tidal volume. This resulted in approximately 30% decreases and increases to the measured pCO2, respectively, as seen in Table 3 and FIGS. 7-9. This linear inverse relationship is a known correlation between ventilation and oxygenation and is vitally important for treatment strategies in caring for those with respiratory damage. The final phase of the animal test was used to bring the animal back to its physiological baseline. Maintaining the tidal volume at 650 mL saw the blood gasses return to their original starting point. This indicates the ventilation was allowing the recovery of the animal to its healthy state and did not induce any unexpected response to the lungs. Additionally, with the venous blood gases showing a congruent return to the baseline values, this gave strong indication of the cardiovascular system also safe from undue damage from the forced, mandatory ventilation. After the procedure, the animal subject was examined, post-mortem, and the lungs showed no gross damage and the X-Rays confirmed that the lungs were kept safe during the experiment, with the ventilator managing to safely ventilate the lungs with no detected damage from this experiment.

The ventilator provided herein represents a novel approach to large scale crisis response for ventilator designs. Typical ventilators require the use of internal compressors and/or bellows and are comprised of hundreds of components. This results in a cost of tens of thousands of dollars (USD) and thus are not in large supply typically, and often require extensive training, and maintenance before they can be deployed. Though very rich in features such as complex, patient-respondent, ventilation modes and monitoring, in a crisis that necessitates a large supply of agile ventilation devices, such as the COVID-19 pandemic, these conventional ventilators are a poor design. The ventilator provided herein represents a new design philosophy for a modern, agile, ventilator that can be readily deployed in locations where a large outbreak of a respiratory disease has occurred. The ventilator provided herein can be stockpiled in large numbers and, due to its minimal feature set, can be expeditiously trained on and deployed to treat those in need of mechanical ventilation assistance. While the initial guidance and recommendations from the FDA at the earlier stages of the COVID-19 pandemic allowed for a wide range of design approaches and looser performance targets, these have been steadily tightened and made more restrictive to ensure safer and longer lasting designs are authorized. Many of the designs that were present in the spring of 2020 have become stale and unfit to the current standards and the ventilator provided herein is one of the few that remain and the only one of its kind to be tested on a large animal prior to FDA authorization.

The present disclosure presents a new, versatile, rapid-response based design of a ventilator for the current COVID-19 pandemic. With such a large number of infected people requiring ventilators, the existing supplies have been overrun and additional, quick to make, efficacious ventilators were required. The ventilator provided herein has been authorized under the FDA's Emergency Use requirements and has been shown to perform the requisite ventilator parameters outlined in the FDA documentation. Furthermore, an animal study was conducted on an adult porcine subject with healthy lungs to observe the real-world fidelity of the relationship between the ventilator settings and the physiological response. Good agreement between the tidal volumes and the oxygenation of the blood was found and the animal responded well to the changes in the ventilator settings. No damage was found to the animal lungs post-mortem and the device was simple and straightforward to use by personnel not highly skilled in ventilation use. With these factors, the ventilator provided herein will be a successful tool in the treatment of large-scale outbreaks of respiratory diseases such as COVID-19

### Conclusion

The present disclosure introduces a low-cost, versatile ventilator designed and built in accordance with the Emergence Use guidance provided by the US Food and Drug Administration (FDA) wherein an external gas supply supplies the ventilator and time limited flow interruption guarantees tidal volume. The goal of this device is to allow a patient to be treated by a single ventilator platform, capable of supporting the various treatment paradigms during a potential COVID-19 related hospitalization. This is a unique aspect of this design as it attempts to become a one-device-one-visit solution to the problem, whereas other published, rapid response devices have focused only on a single type of ventilation. The design philosophy of the ventilator is based on a continuous flow, Pressure Limited Time Cycled format and the parameters of the device are tested in accordance with the FDA's Emergency Use Authorization requirements. Additionally, to test the device on a living subject, a pig is sedated and placed on mandatory ventilation while the ventilator controls are adjusted to bring the animal to both hypoxic and hyperoxic states until it was brought back to a healthy baseline. This test was used to detect risk of lung injury and after a post mortem, the lungs were found to be well protected by the ventilator during this procedure. The ventilator provided herein has shown to be a promising candidate for emergency use during the COVID-19 pandemic and beyond in cases where a rapid-response and versatile ventilation platform are needed.

### EXAMPLE 1

A surge in mechanical ventilator need may occur during mass casualty or pandemic scenarios. In these circumstances there is a need to rapidly produce low cost mechanical ventilators at scale.

Current mechanical ventilator availability supports the provision of healthcare services for critically ill individuals under usual circumstances; however, the healthcare system is ill-prepared to manage surge scenarios where the need for mechanical ventilation may dramatically exceed available ventilator capacity.

The systems and methods disclosed herein will allow for the rapid production of a low cost mechanical ventilator.

The present disclosure provides an exemplary arrangement of key mechanical parts, sensors, and actuators of a low-cost ventilator that is designed to be deployed in large quantities. All parts displayed inside the “Ventilator” box are intended to be provided as part of the delivered system while parts that are outside of the box are required for operation but assumed to be on hand at the hospital where this ventilator will be deployed. Electronic components including a user interface are not shown in this schematic, but will be included in the delivered system. A 120V AC power supply will be required for operation of this system.

In another embodiment, a continuous flow design architecture, there is a continuous flow design which minimizes the amount of components, and therefore reduces complexity and points of failure, by working on the principle of a constant flow of a mixed air/oxygen gas mixture coming into the system. This differs from the regular design where a reservoir is often used to create and store a gas mixture which is produced through the consumption of multiple constituent gases. This design is shown in the schematic that is included in this overall submission. This design relies on a pre-mixed, constant or variable flow of single or multiple gases. In yet another embodiment, a low cost design based on a single flow-manifold and custom flow sensors and actuators is contemplated. The mass production of low-cost ventilators based on the same flow diagram philosophy. However this will not be from consumer-available parts but instead be created via custom low-cost sensors, and injection molded components. Notably these sensors will not draw on the limited supplies of off-the-shelf flow meters and flow totalizers; instead we have incorporated floating ball flowmeters and spirometers into a simple, molded part set.

In yet another embodiment, a clamshell and/or single manifold construction eliminates tubing connections between ventilator elements, instead incorporating the elements and their connections into a few parts. The simplicity of this approach will lead to more consistent manufacturing, particularly as the project ramps production.

In yet another embodiment, an additional sensor is on the body of the manifold to detect if it is upright. The spirometer puck requires gravity to return to its resting position after inspiration or expiration. If the spirometer is not vertically oriented, it may not function properly. Therefore, a sensor can be used to detect gravitational orientation of the spirometer and provide an alarm if excessive angulation of the spirometer is detected. Preferred embodiments of the gravitational sensor include a MEMS accelerometer, a plumb bob, an air bubble level, a mercury tilt sensor, or a rolling ball.

In another embodiment, the gasses flowing through the ventilator are measured with both a ball flowmeter and a spirometer. These measurements are compared to continuously check the accuracy of the ventilator. Using two different physical principles to measure the same value of flow rate increases the reliability of the measurement.

In another embodiment, the input valve also functions as a vent during exhalation. This allows the use of an air/oxygen mix from a standard blender, already widely used in hospitals.

In another embodiment, one or more cameras watches the flow meters and spirometry to measure their position and record flow data.

### Ventilation Modes

### Mandatory Ventilation

In Mandatory Ventilation mode, the ventilator will automatically deliver breaths to the patient based on software controlled timing. The ventilator operator will specify the breaths per minute through the user interface which will dictate the frequency at which the breaths are initiated. The user will also be able to specify the inspiratory/expiratory (I:E) ratio and the target volume of each breath. The I:E ratio will determine the amount of time during each breath that inhalation occurs. If the target volume is delivered before the inspiratory time is complete, the ventilator will hold the breath by sealing off the inspiratory and expiratory limbs of the circuit. The state of the two actuated valves in the system is indicated in Table 4.

### Assisted Ventilation

In assisted ventilation mode, the ventilator delivers a breath when it is triggered by the patient. Breaths are triggered when a patient attempts to inhale, which causes a pressure decrease in the patient circuit, which is detected by the patient airway pressure sensors. In assisted ventilation mode the patient dictates the breaths per minute and the time for each breath may not be constant, so the ventilator operator specifies the inspiratory time rather than the I:E ratio. The target volume is also specified by the operator in this mode. The expiratory time lasts from the end of the inspiratory time to the next time the patient triggers a breath.

The same valve states shown in Table 4 above apply during assisted ventilation.

A maximum breath time will also be specified. If that time threshold is exceeded, the ventilator will deliver a mandatory breath with the specified inspiratory time and wait for another breath to be triggered.

### Inspiratory Hold

During either mandatory or assisted ventilation, the operator may initiate inspiratory hold via a button on the user interface. The operator must hold the button down in order to initiate this event. While the button is being held down, a complete breath will be delivered and then the inspiratory and expiratory limbs of the patient circuit will be sealed off. When the button is released the ventilator continues operation with a normal exhalation.

### Additional Considerations

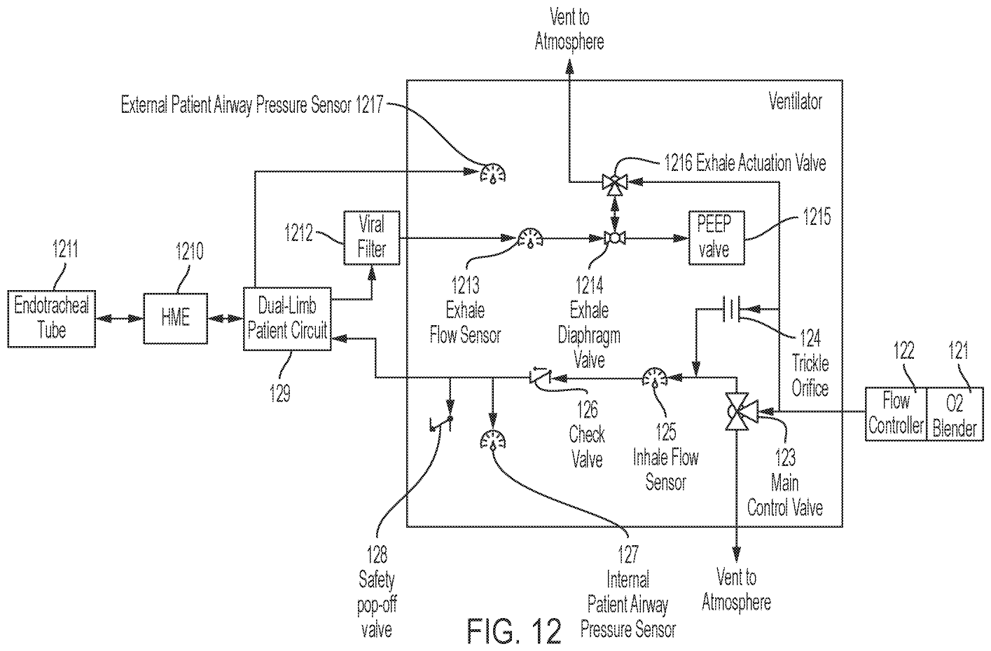

FIG. 12 shows an exemplary device. The system components include, but are not limited to: an O2 blender (121) is required in order to deliver a mixture of air and oxygen to the system. It is assumed that the intake of the blender will be 50 PSI sources of air and oxygen. A flow controller (122) will be required to allow the user to reduce the pressure at the output of the oxygen blender and set the flow rate of gas delivered to the patient. A flow controller with an integrated Thorpe tube flowmeter is recommended. The main control valve (123) is a two-way valve that switches the ventilators gas inlet between the patient circuit and venting to atmosphere. When a breath is delivered to the patient, the intake case is directed to the patient circuit. When inhalation is complete, the main control valve diverts the intake gas to atmosphere so that excessive pressure does not build up in the intake line. The trickle orifice (124) allows a small amount of gas to bypass the main control valve. This provides a constant stream of gas at a much lower flow rate than what is delivered during inhalation. This stream allows the ventilator to maintain a non-zero PEEP during operation and prevents CO2 buildup in the patient circuit. The inhale flow sensor (125) measures the flow of all gas that is delivered to the patient. The integration of the signal from this sensor allows calculation of volume delivered and provides the capability for a volume vs time or flow vs time plot to be displayed to the operator. The check valve (126) prevents the patient from pushing contaminated air into the ventilator circuitry during exhalation. This internal patient airway pressure sensor (127) will detect pressure in the patient airway which. At all times, if the patient airway pressure is higher or lower than values specified by the operator an alarm will sound. During assisted ventilation a sufficiently negative reading on this sensor relative to the PEEP value will trigger an assisted breath. The safety pop-off valve (128) is a purely mechanical valve which will vent the patient circuit to atmosphere if the pressure in the patient circuit becomes higher than what is safe. This valve will be purely mechanical so that any failure of the electronics or software that cause a high patient airway pressure can be mitigated. The patient circuit (129) is assumed to be provided by the hospital. A circuit with a port to measure pressure as close to the patient as possible is recommended. A Heat and Moisture Exchanger (HME) (1210) is recommended for use with this ventilator, but is not included in the delivered system. The endotracheal tube (1211) delivers the air to the patient's lungs. It is not included in this system. It is recommended that a viral filter (1212) is connected to the expiratory limb of the patient circuit and replaced periodically. These filters will not be provided with this system. The exhale flow sensor (1213) will be identical to the inhale flow sensor and will measure the flow of exhaled gas. This will enable calculation of exhaled volume of each breath and can be used to detect breath stacking or leak conditions. The exhalation diaphragm valve (1214) allows air to exit the expiratory limb of the patient circuit during exhalation. The diaphragm is connected to the pressurized intake gas during inhalation so that the expiratory limb is blocked. During exhalation the diaphragm is vented to atmosphere so that the gas in the patient circuit can open the valve and exit. The PEEP valve (1215) is a spring-loaded mechanical valve that resists the exhalation pressure in the patient circuit. The spring force can be set to achieve a desired PEEP value. The exhale actuation valve (1216) is a two-way valve that switches the exhale diaphragm between atmosphere and intake gas pressure. During inhalation this valve connects the diaphragm to intake gas so that it can be pressurized and prevent air in the circuit from escaping through the expiratory limb. During exhalation this valve connects the diaphragm to atmosphere. The External Patient Airway Pressure Sensor (1217) sensor is connected to a line that runs to a port on the patient circuit in order to measure the pressure in the patient circuit as close to the patient as possible. The ability to measure this pressure allows for detection of leaks and blockages in the patient circuit and gives a more accurate estimation of the patient airway pressure than measuring this pressure inside the ventilator.

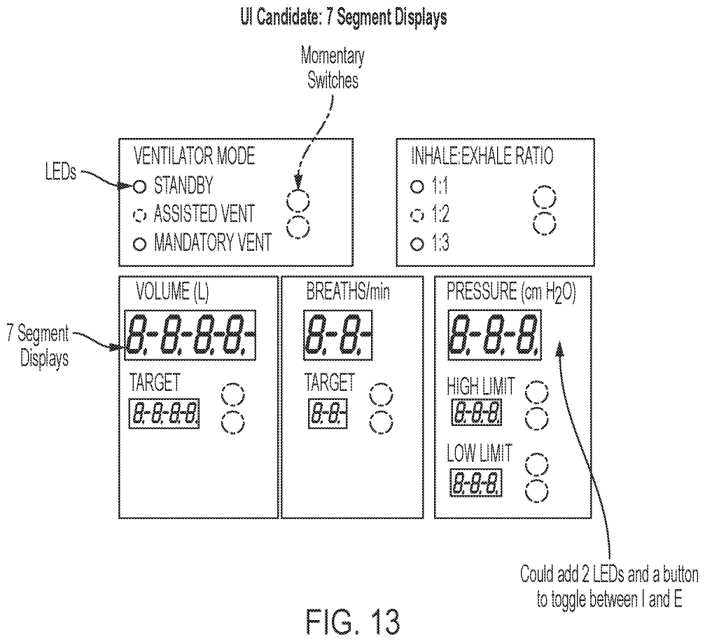

FIG. 13 depicts an exemplary control interface that allows the user to select ventilator mode, inhalation to exhalation ratio, tidal volume, and pressure settings.

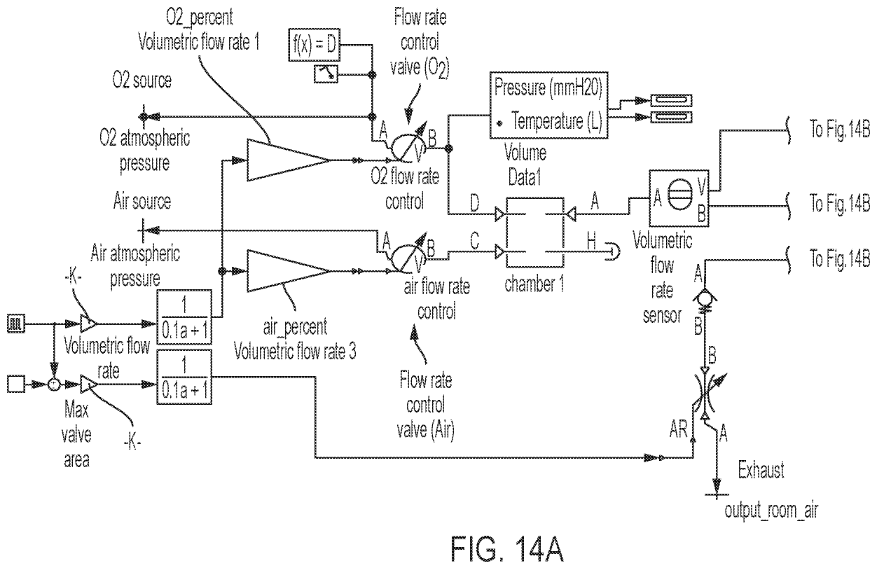

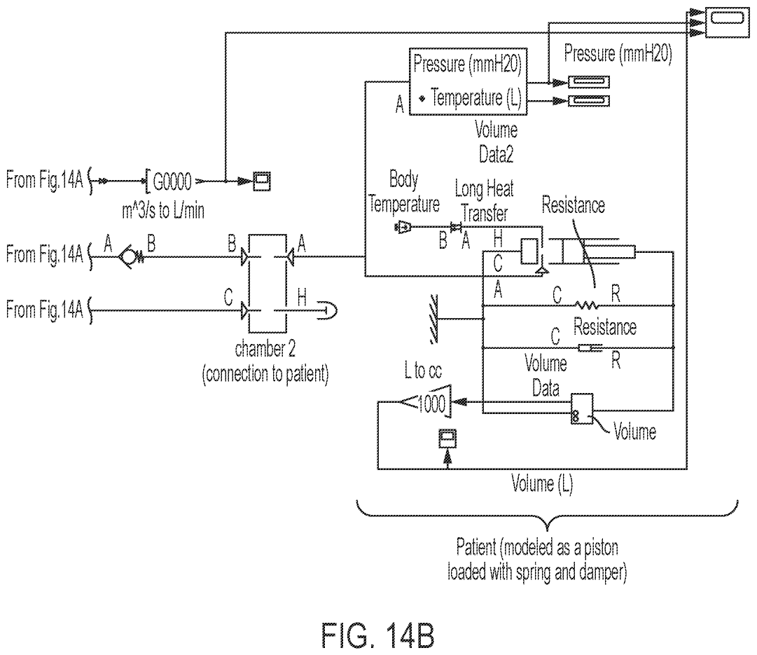

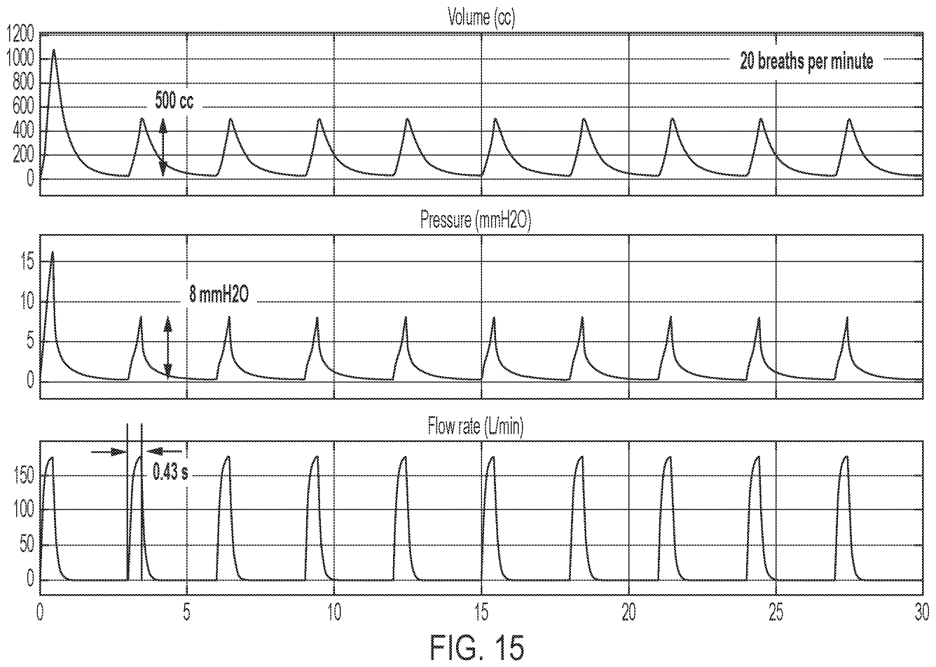

FIGS. 14 and 15 show the structure of this system using a computational model to observe the response and allow for interaction with the ensemble of components under prescribed input. Specifically, FIG. 14 shows the schematic of the system, while FIG. 15 shows an example response of the system's output variables during normal operation.

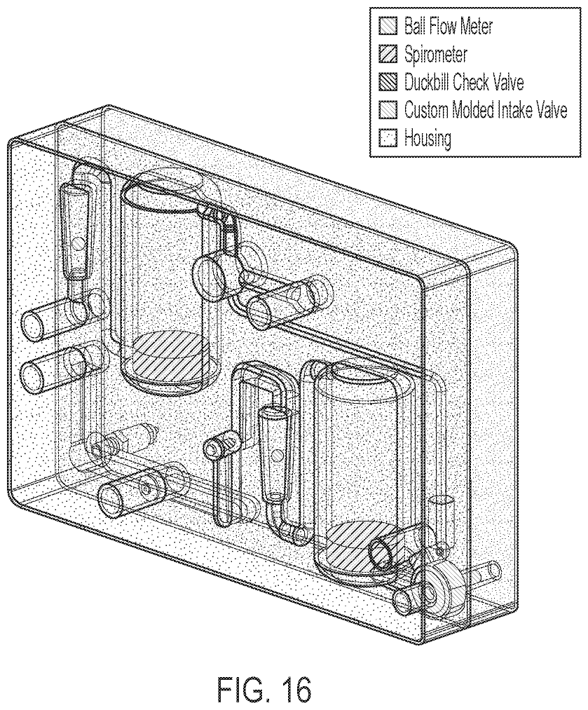

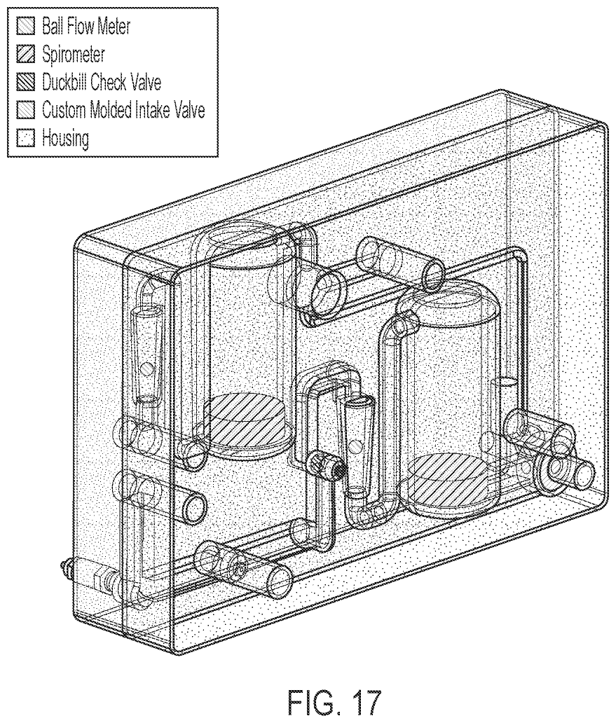

FIGS. 16 and 17 illustrate an exemplary device with inhalation and exhalation spirometers, ball flow meters, custom molded intake valve, duckbill check valve, and housings that are molded and incorporated in all plumbing shaded as indicated in the key.

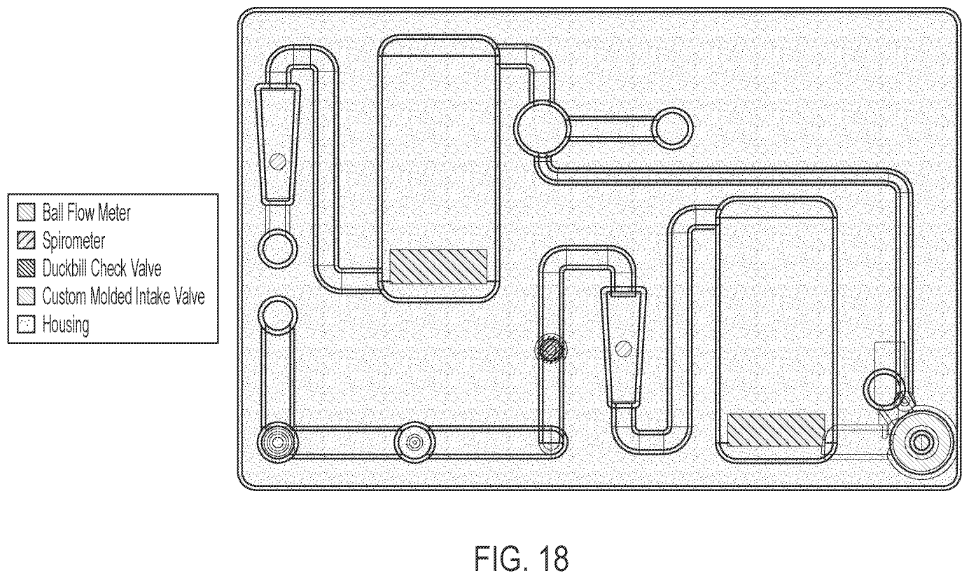

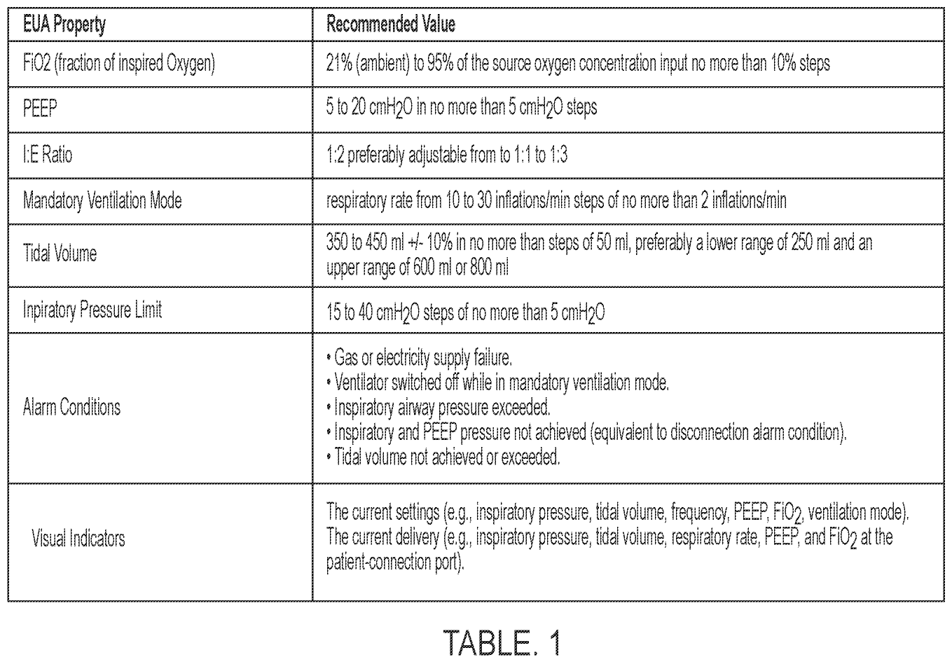

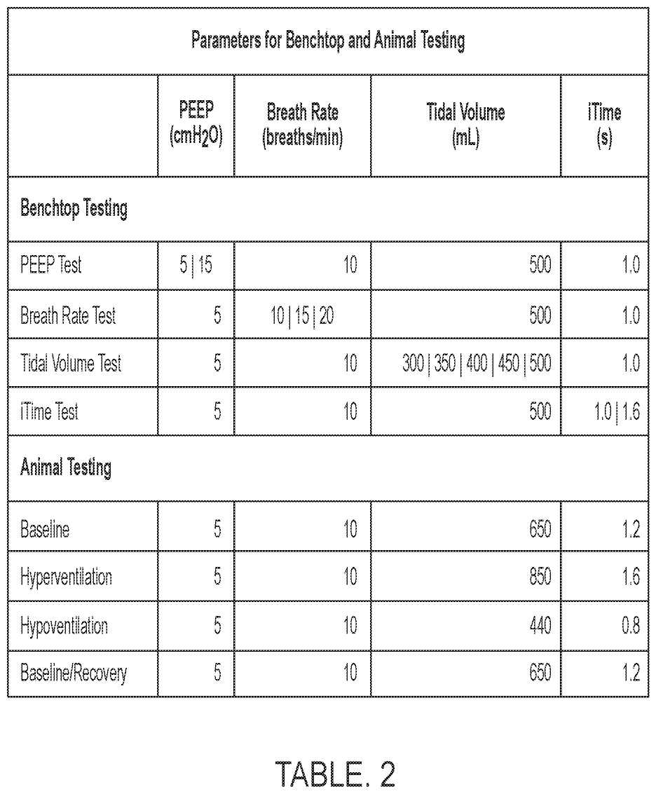

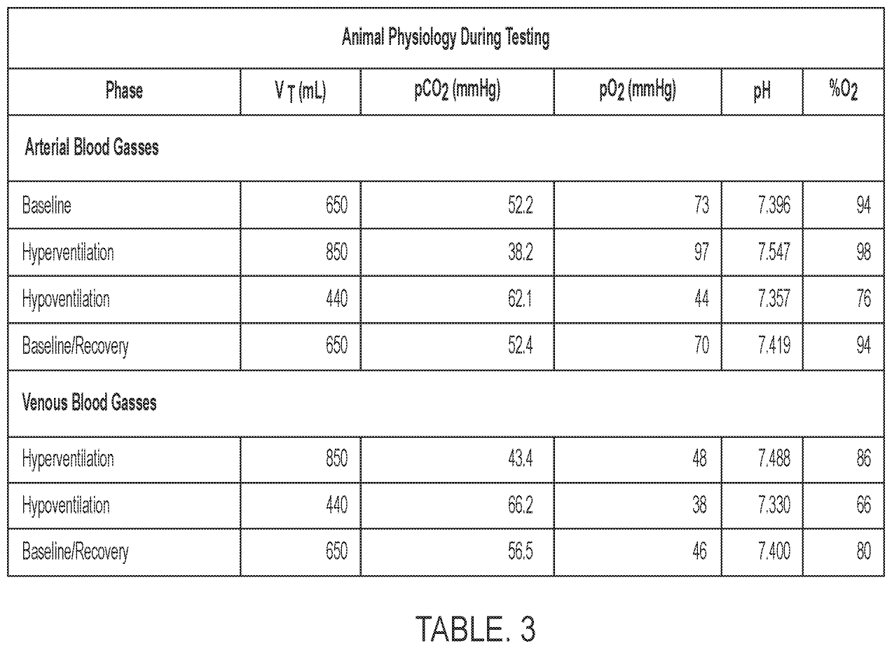

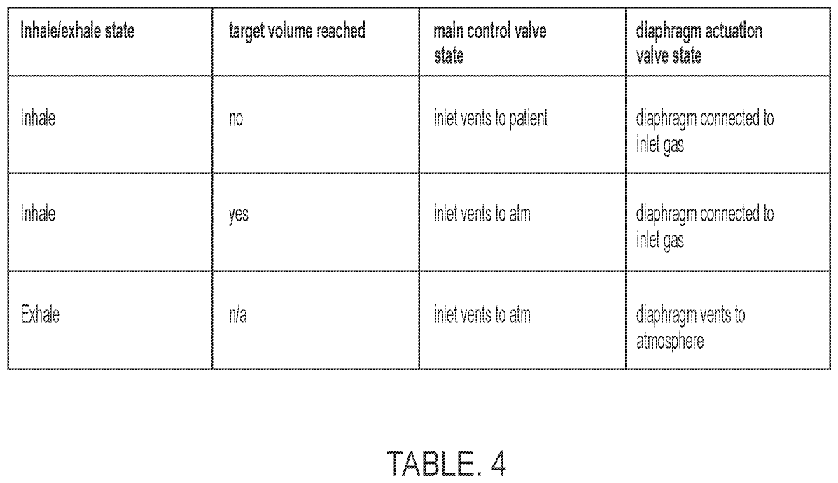

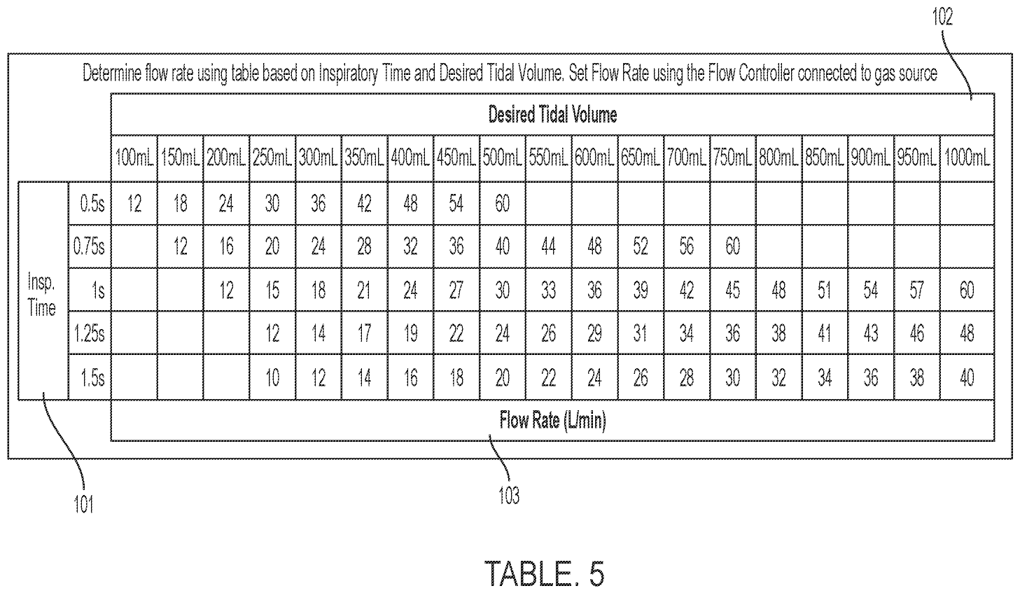

FIG. 18 illustrates the plumbing and gas flow path connecting the elements shown at FIGS. 16 and 17.

### Example 2

A surge in the need for ventilator and ventilator parts may occur during mass casualty and/or pandemic scenarios. In these circumstances there is a need to rapidly produce low cost ventilators that are not supply-chain restricted.

The availability of ventilators is currently targeted to meet the demands of critically ill individuals under normal circumstances. The healthcare system is not prepared to manage a surge in need of ventilators, this would mean the demand for ventilators would exceed the current supply capacity.

The systems and methods provided herein will allow for a ventilator that does not have means to sense flow volume to still deliver the desired volume.

The present disclosure provides the arrangement of key parameters, layout, and metrics, of a ventilator flow rate table (shown at Table 5) that can be used on a ventilator with minimal or no volume monitoring. The inspiration times and desired volumes to be delivered are used as the vertical and horizontal axes, respectively, and the required flow rate that shall be set for the incoming gas occupy the interior of the table.

The present disclosure provides a method in which the volume delivered by the ventilator can be set by the user adjusting non-volume parameters. Non-volume parameters include, but are not limited to gas flow rate, pressure, inspiratory time, inspiratory:expiratory ratio, and respiratory rate. The desired volume is achieved when the user refers to a table or tables, such as Table 5 that may or may not be affixed to the ventilator whose X and Y axes and individual fields have, in any combination, the non-volume parameter(s) and the desired volume. By referring to a table such as Table 5, the user can identify the settings for the non-volume parameters and set the ventilator to the desired values.

Table 5 shows an exemplary flow rate table. The table components include, but are not limited to: a list of inspiratory times (101) on the vertical axis, a list of desired delivered volumes (102) on the horizontal axis, and the interior of the table is populated by the volumetric flow rate (103), in L/min required to be supplied to the ventilator to achieve the desired delivered volume for the chosen inspiratory time.

What is claimed is:

1. A ventilator system configured to switch between one or more invasive ventilation modes and one or more non-invasive ventilation modes, the ventilator system comprising:
an externally pressurized source of pre-mixed gas including air and oxygen;
one or more inspiratory valves configured to deliver incoming pre-mixed gas to a patient's breathing circuit; and
one or more expiratory valves configured to remove outgoing gas from the patient's breathing circuit;
wherein in the one or more invasive ventilation modes, the inspiratory valves and the expiratory valves are configured to open and close to allow or prevent the passage of gas as needed in order to enforce a respiration cycle within the patient; and
wherein in the one or more non-invasive ventilation modes, the inspiratory valves and the expiratory valves are kept open in order to allow the gas to pass freely through the system.

2. The ventilator system of claim 1, wherein the respiration cycle is based on a pressure-limited-time-cycled breathing loop.

3. The ventilator system of any of claim 1 or 2, further comprising a manual control valve, wherein a flow rate is set by a user via the manual control valve.

4. The ventilator system of any of claims 1-3, wherein the manual control valve is a Thorpe Tube.

5. The ventilator system of any of claims 1-4, wherein an inspiratory time is set by a user such that a known volume of gas is delivered during each inspiration phase.

6. The ventilator system of any of claims 1-5, further comprising one or more sensors positioned on the expiration side of the patient's breathing circuit and configured to measure exhaled volume of gas.

7. The ventilator system of claim 6, wherein the one or more sensors include one or more of a flowmeter or a spirometer.

8. The ventilator system of any of claims 1-7, further comprising a monitoring device configured to monitor pressures in the inspiratory valves and the expiratory valves to identify instances of leaks or blockages.

9. The ventilator system of any of claims 1-8, wherein switching between the one or more invasive ventilation modes and the one or more non-invasive ventilation modes is controlled based on input from a user.

10. A method, comprising:
delivering, by a ventilator, incoming pre-mixed gas including air and oxygen to a patient's breathing circuit via one or more inspiratory valves of the ventilator;
removing, by the ventilator, outgoing gas from the patient's breathing circuit via one or more expiratory valves of the ventilator; and
switching, by the ventilator, between one or more invasive ventilation modes and one or more non-invasive ventilation modes;
in the one or more invasive ventilation modes, opening and closing, by the ventilator, the inspiratory valves and the expiratory valves to allow or prevent the passage of gas as needed in order to enforce a respiration cycle within the patient; and
in the one or more non-invasive ventilation modes, opening, by the ventilator, the inspiratory valves and the expiratory valves in order to allow the gas to pass freely through the system.

11. The method of claim 10, wherein the respiration cycle is based on a pressure-limited-time-cycled breathing loop.

12. The method of claim 10 or claim 11, further comprising receiving, by the ventilator, a flow rate set by a user via a manual control valve of the ventilator.

13. The method of any of claims 10-12, wherein the manual control valve is a Thorpe Tube.

14. The method of any of claims 10-13, further comprising setting an inspiratory time such that a known volume of gas is delivered during each inspiration phase.

15. The method of any of claims 10-14, further comprising measuring, by the ventilator, an exhaled volume of gas via one or more sensors of the ventilator positioned on the expiration side of the patient's breathing circuit.

16. The method of claim 15, wherein one or more sensors include one or more of a flowmeter or a spirometer.

17. The method of any of claims 10-16, further comprising monitoring, by a monitoring device of the ventilator, pressures in the inspiratory valves and the expiratory valves to identify instances of leaks or blockages.

18. The method of any of claims 10-17, wherein switching between the one or more invasive ventilation modes and the one or more non-invasive ventilation modes is controlled based on input from a user.
